(19) United States
(12) Patent Application Publication
(10) Pub. No.: US 2021/0131998 A1
(43) Pub. Date: May 6, 2021

(54) GAS SENSOR NANOCOMPOSITE MEMBRANES

(71) Applicants: ROYAL MELBOURNE INSTITUTE OF TECHNOLOGY, Melbourne (AU); MEAT & LIVESTOCK AUSTRALIA LIMITED, North Sydney (AU)

(72) Inventors: Kourosh KALANTAR-ZADEH, Melbourne (AU); Kyle BEREAN, Melbourne (AU); Nam HA, Melbourne (AU); Jian Zhen OU, Chadstone (AU)

(73) Assignees: ROYAL MELBOURNE INSTITUTE OF TECHNOLOGY, Melbourne (AU); MEAT & LIVESTOCK AUSTRALIA LIMITED, North Sydney (AU)

(21) Appl. No.: 17/083,465

(22) Filed: Oct. 29, 2020

Related U.S. Application Data
(63) Continuation of application No. 15/507,527, filed on Feb. 28, 2017, now abandoned.

(51) Int. Cl. G01N 27/407; G01N 27/40; B82Y 30/00; A61B 5/145; A61B 5/07; B01D 67/00; B01D 69/02; B01D 69/14; A61B 5/00; B01D 53/22

(52) U.S. Cl. CPC G01N 27/407; A61B 5/01; B82Y 30/00; A61B 5/14539; A61B 5/14542; A61B 5/073; B01D 67/0079; B01D 69/02; B01D 69/141; A61B 5/6861; B01D 53/228; B01D 69/148; B01D 69/147; B01D 53/22; G01N 27/40

(57) ABSTRACT

A gas permeable, liquid impermeable membrane for use with gas sensors consists of a film forming polymer which incorporates nanoparticles selected to improve one or more of the following: permeability to gases, to selectively regulate permeability of selected gases through the membrane, to inhibit microbial growth on the membrane. A capsule shaped container consists of wall material biocompatible with a mammal GI tract and adapted to protect the electronic and sensor devices in the capsule, which contains gas composition sensors, pressure and temperature sensors, a microcontroller, a power source and a wireless transmission device. The microprocessor receives data signals from the sensors and converts the signals into gas composition and concentration data and temperature and pressure data for transmission to an external computing device. The capsule wall incorporates gas permeable nano-composite membranes with embedded catalytic and nano void producing nanoparticles, enhancing the operation, selectivity and sensitivity of the gas sensors.

This invention relates to nano-composite membranes for use with gas sensors to enhance the performance of the gas sensors in terms of selectivity, response time and durability. These membranes are particularly useful in an ingestible sensor capsule for monitoring gases generated in the gastrointestinal (GI) tract of mammals including humans.

### BACKGROUND TO THE INVENTION

While there are currently diagnostic tools available such as capsule endoscopy and breath analysers, there is no equipment for the analysis of the gas constituents in the gastrointestinal tract. There are many reports on the strong likelihood of the association of these gas constituents to different illnesses. However, due to lack of any suitable tool and the inconveniences that these measurements create for the patients, the potential of this area has yet to be fully realized.

USA patent 8469857 discloses a method of diagnosing GI conditions by analysing gases in breath analysis.

Patent application WO2013/003892 discloses a capsule with gas sensors and a gas permeable membrane for use with ruminant animals.

USA patent application 2009/0318783 discloses a computerised method analysing data from the GI tract using an ingestible capsule that contains a sensor and providing data on the measurement plotted against time.

USA patent application 2013/0289368 discloses an ingestible capsule with a gas detector to assist in diagnosing diseases of the GI tract.

One difficulty in using devices described in the prior art is the use of membranes without any modification. Even the most non-selective and gas permeative membranes such as polydimethylsiloxane (PDMS) have a long response time. This sometimes reaches several 10s of minutes. For a less dynamic scenario such as placing the capsule inside the rumen of cattle [Patent application WO2013/003892], this may be sufficient. However, for measurements of gas constituents in gastrointestinal (GI) tract, especially in human and other mammals with similar digestive systems, such response times are inadequate.

Another difficulty with prior art devices is the lack selectivity of the pure membranes. For, instance, a pure PDMS membrane allows all gas species to permeate through. This may be acceptable when highly selective gas sensors are used. However, most available gas sensors are non-selective. For instance the current hydrogen (H₂) gas sensors are also sensitive to other gas species such as methane (CH₄). Such lack of specificity seriously compromises the accuracy of the measurements. Another problem of non-selectivity of the pure membranes is possibility for the permeation of highly acidic gas species (such as those in digestive systems including hydrogen sulphide (H₂S) and oxides of nitrogen (NOₓ) and exposure of sensor bodies to them. This significantly reduces the life time of sensors. For instance, most of the commercial H₂ gas sensors, under exposure to 100 ppm H₂S gas fail to operate after just 24 hours.

Another challenge is the colonization of foreign microorganisms, such as microorganisms of the digestive system onto the surface of the membrane. Pure membranes such as PDMS show a slight antimicrobial effect. However, this is not sufficient to stop the colonization of the microorganisms of the digestive tract for relatively long time. For instance, a capsule operation for measurement of gas species of GI tract requires more than a day up to several weeks.

It is an object of this invention to provide nano-composite membranes that enhance the performance of gas sensors.

It is an object of this invention to ameliorate the prior art problems with sensor capsules and provide a more effective and responsive sensor capsule for use in the digestive system.

### BRIEF DESCRIPTION OF THE INVENTION

To this end the present invention provides a gas permeable, liquid impermeable membrane for use with gas sensors in which the membrane consists of a film forming polymer which incorporates one or more nanoparticles selected to improve one or more of the following:
the permeability to gases, to selectively impede or exclude permeation by some gases while facilitating the passage of selected gases through the membrane, to inhibit microbial growth on the membrane.

These membranes may be used in any application where the response time and sensitivity of gas sensors needs improvement. The membranes of this invention were developed to address these problems including those encountered in sensing gases within the mammalian digestive and gastrointestinal systems.

The unique feature of the membranes of this invention are several key functionalities that enhance the performance of gas sensors to the levels required for accurate gas constituent measurements during the life of the sensor. The nano-composite membranes allow high selectivity passage of desirable gas species to gas sensor arrays, block unwanted interfering gas species, and stop the colonization of undesirable microorganisms on the surface of the membranes.

The membranes are preferably selected from gas permeable liquid impermeable polymeric materials which are either glassy or rubbery polymers. Examples of glassy polymers used consistently in industrial applications include; polyimides, polyarylates, polycarbonates, polysulfones, cellulose acetate, poly (phenylene oxide), polyacetylenes and poly [1-(trimethylsilyl)-1-propyne] (PTMSP). In comparison, rubbery polymers that are of industrial relevance are less diverse, with poly (dimethylsiloxane) being the most prominent.

The membranes used in this invention are polymeric nano-composite membranes with incorporated nano-materials, with several possible functionalities.
1. They may be able to function as reactors embedded into the polymeric matrix. These nano reactors reversibly or non-reversibly interact with materials on the surface, penetrate within and/or passing through the body of the membranes to convert them into other materials.
   The nano-reactors may be used for enhancing gas and liquid separation and permeation of the membranes: (1) enhancing selectivity and sensitivity of the membranes to specific has or liquid molecules, ions, atoms and other particles, (2) enhance the separation efficiency of gas and liquid species using the membranes, and (3) reactively manipulate the gas or liquid molecules, ions and toms that pass through the membrane to obtain a product.
   These membranes are made of highly permeable polymers such as polydimethylsiloxane (PDMS), polyacetylene, poly(l-trimethylsilyl-1-propyne) (PTMSP). Some other well-known families of these polymers include perfluoropolymers, poly(norbornene)s and polyimides. Embedding nanoparticles of materials such as metal oxides or chalcogenides (e.g. ZnO, In₂O₃, WON, TiO₂, WS₂, MoS₂, . . . ), other semiconductors, metals (e.g. Ag, Au, Pt, . . . ), carbon based materials (e.g. graphene, carbon nanotubes, . . . ) as well as other nanomaterials especially catalytic nano materials. These materials catalyze the gas or liquid species of interest inside the GI tract, at the body temperature, without themselves participating in the interaction. Some of the most suitable nanomaterials are well known catalytic metals including Ag, Au, Pt and Pd and materials with a relatively small band gap such as MnO₂ and FeOₓ, CuOₓ, WS₂ and MoS₂.
2—Many of the above-mentioned nanomaterials may also show antimicrobial capabilities at very low concentrations. Materials such as Ag, MnO₂, Pt and Au can significantly reduce the chance of microorganism colonization on the surface of the membranes at or near room temperature and to much higher temperatures. This hence increases the lifetime of the capsule.
3—The third possible functionality of nanomaterials is that they give the desired structure to the nano-composite. Incorporating selected nanofillers into the structure of polymers adds extra degrees of freedom to work with in order to satisfy the permeability and selectivity conditions at the same time. Embedding nano-fillers within a polymer can adjust the solubility of gas species, systematically manipulate the polymeric chain molecular packing, producing extra interfacial voids or areas around the nanofillers and change the asymmetry. The formation of nano voids can especially help in increasing the permeability. The surface diffusivity of gas molecules is much faster than the permeation within the bulk of the membrane. As a result, if using a nanomaterial the surface area within the bulk can be increased then the overall permeation for the selected gas increases.
Materials such as graphene and carbon nanotubes may form nano-frameworks for increasing the surface area. The gas permeation in the membranes with such frameworks may increase by an order of magnitude.

The following table sets out the functionalities that may be achieved.

Nanomaterial and their Effects on Polymeric Compounds

| | Effect when | |
|---|---|---|
| | embedded in | |
| | polymeric | |
| Type of nanomaterial | compounds | Examples |
| Metallic | Catalytic, antimicrobial | Gold, silver, platinum, |
| | | palladium, . . . |
| Metal oxide | Catalytic | MnO₂, WO₃, CuₓO, |
| compounds | | FeOₓ . . . |
| Transition metal | Catalytic, produces | MoS₂, WS₂, WSe₂, . . . |
| chalcogenide | nanovoids | |
| compounds | | |
| Carbon based | Producing nanovoids, | Graphene, carbon |
| nanomaterials | high affinity to | black, carbon |
| | hydrogen (block it) | nanotubes, bucky balls |
| Conventional III-IV | Catalytic properties, | CdS, CdSe, . . . |
| semiconductors | changing selectivity | |

In another aspect this invention provides a capsule adapted to be introduced into the digestive system and GI tract of a mammal which consists of capsule shaped container consisting of a wall material capable of being bio compatible with the digestive system and being adapted to protect the electronic and sensor devices contained in the capsule;

said capsule containing an array of gas composition sensors, pressure and temperature sensors, a micro controller, a power source and a wireless transmission device;

said capsule wall incorporating gas permeable membranes adjacent said gas sensors which incorporate nanoparticles which facilitate the operation, selectivity and sensitivity of the gas sensors;

the microprocessor being programmed to receive data signals from the sensors and convert the signals into gas composition and concentration data and temperature and pressure data suitable for transmission to an external computing device.

The unique feature of this capsule is the implementation of nanoscomposite membranes along with the array of gas sensors that significantly enhances the performance of the gas sensor array in terms of response time, selectivity and durability.

The gas sensor capsule allows an accurate identification of the target gases in situ, where they are produced, and assists in linking them with more certainty to the state of health and the presence of illnesses. These capsules permit the whole gastrointestinal tract to be surveyed, not just the accessible parts. In addition, the procedure is non-invasive and capsules pass out of the body of the subjects at the end of the process.

Especially for human applications, after being swallowed, the “gas sensor capsule” will help gastroenterologists to survey human subjects' gas species and their concentrations in the oesophagus, stomach, small intestine parts (duodenum, jejunum and ileum), caecum and large intestine. The capsule may also help in understanding the gas species produced in other mammalians and associated them with their diets, state of health and the volume of gas production (for gas mitigation or productivity efficiency assessments). The device allows the possibility of accurately investigating and fully obtaining the correlations between the existing gas species and gastrointestinal medical illnesses. Establishing such correlations and accurately assessing the gas content of the digestive tract of individual subjects will help to reveal the effects of the existing microorganisms in the digestive tract and help prescribing correct medications, resulting in more accurate targeting of gastrointestinal illnesses. As such, the gas sensor capsule will be an invaluable tool for assessing health status using non-invasive diagnostics.

The gas sensor capsule with nano-composite membranes of this invention is a diagnostic and monitoring tool, which may be swallowed and has the capability of accurately sampling gas constituents throughout the entire gastrointestinal tract. Its advantages are:

1—The nano-composite membranes allow for high selectivity and sensitivity measurements of gas constituents along the tract.
2—The membranes are designed to be highly permeable to the gas species of interest (ideally to be transparent to the selected gas) as a result they reduce the response time of the system for the gas measurements to that of the response time of the array of sensors.
3—The catalytic properties of the nano-composite membranes allow for the longevity of the gas sensor elements protecting them from unwanted caustic gases and vapours.
4—The antimicrobial properties of the nano-composite membranes inhibit the colonization by microorganisms onto the nano-composites and keep the surface clean for a longer time. The nanoparticles also prohibit the blockage of the gas permeable membrane for the duration of the measurement.

Especially for human applications, after being swallowed, the “gas sensor capsule” will help gastroenterologists to survey human subjects' gas species and their concentrations in oesophagus, stomach, jejunum duodenum, ileum, caecum and large intestine. The capsule may also help in understanding the gas species produced in other mammalians and associated them with their diets, state of health and the volume of gas production (for gas mitigation or production efficiency increase). The device allows the possibility of accurately investigating and fully obtaining the correlations between the existing gas species and gastrointestinal medical illnesses. Establishing such correlations and accurately assessing the gas content of the digestive tract of individual subjects will help to reveal the effects of the existing microorganisms in the digestive tract and help prescribing correct medications, resulting in more accurate targeting of gastrointestinal illnesses. As such, the gas sensor capsule will be an invaluable tool for assessing health status using non-invasive diagnostics.

### DETAILED DESCRIPTION OF THE INVENTION

Preferred embodiments of the invention will be described with reference to the drawings in which:

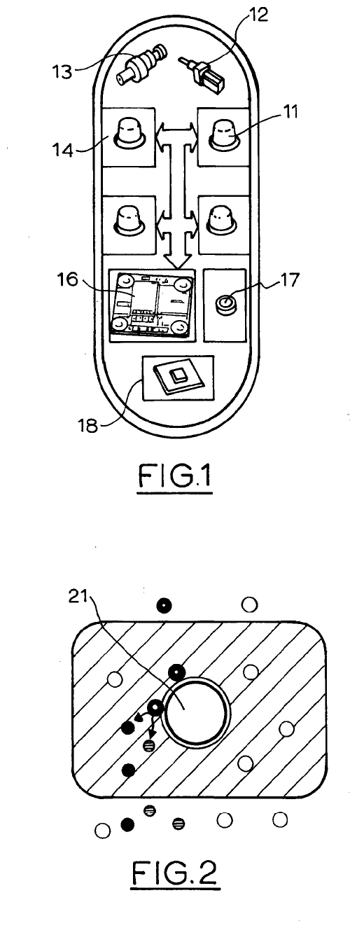

FIG. 1 is a schematic of a preferred capsule of this invention;

FIG. 2 is a schematic illustration of the function of catalytic nano-membranes of this invention;

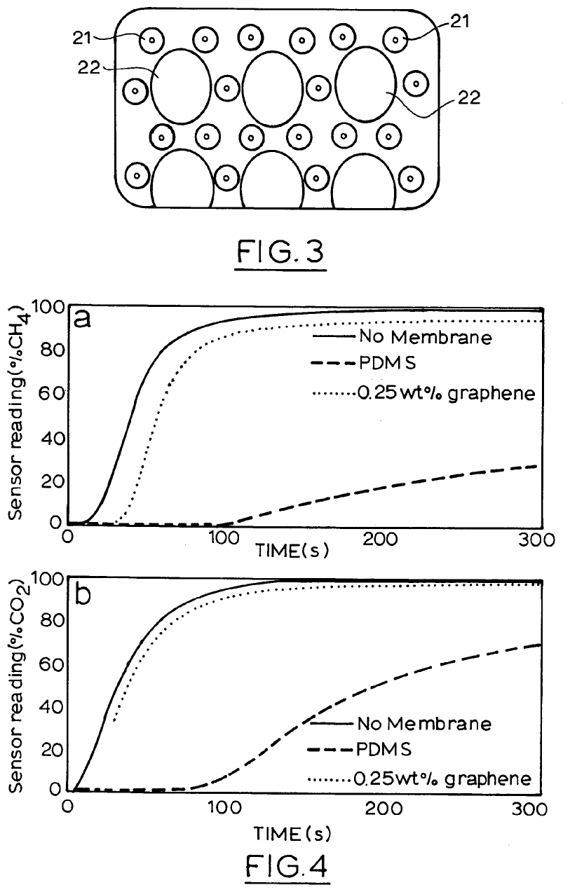

FIG. 3 is a schematic illustration of the nano-voids produced by nano-materials in the membranes of this invention;

FIG. 4 is a graphical illustration of permeation results with membranes according to this invention;

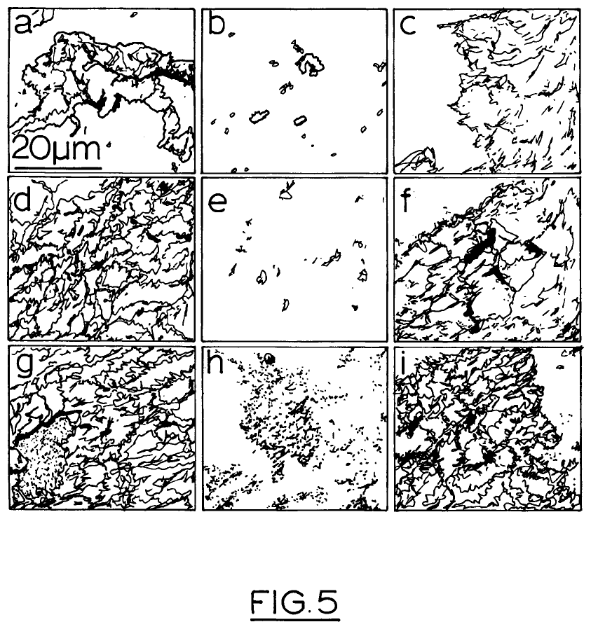

FIG. 5 is a micrograph illustrating microbial growth on membranes;

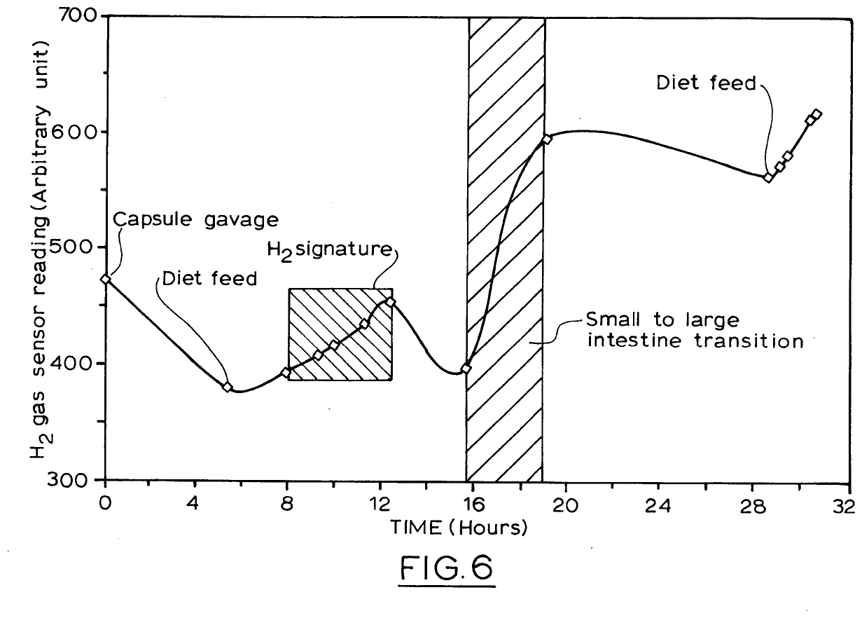

FIG. 6 illustrates the capsule measurement in a pig;

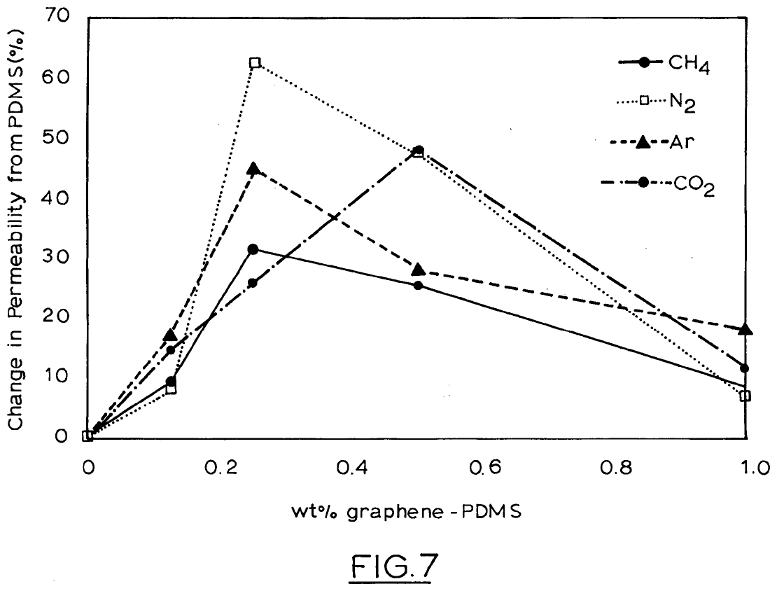

FIG. 7 illustrates change in permeability for each gas species with respect to the change graphene concentration.

The main components of a preferred capsule are illustrated in FIG. 1. The main components are:
Sensors: gas sensors 11 such as CH₄, H₂, CO₂, NOₓ and H₂S as well as volatile organic compound sensors, such as butyrates and acetates, are the main components. These gas species are the most common materials associated with the gastrointestinal tract micro-organisms and have suggested links to specific human health conditions. In addition other sensors including temperature 12 and pressure sensors 13 (also possibly pH sensors) are preferably included to provide environmental information for the gas analysis.
Nano-composite permeable membranes 14 with embedded catalytic 21 and non catalytic nanomaterials (that make structural nano-voids 22): the membranes 14 on the capsule cover allow the passage of certain gas species and catalytically interact with other ones to block them. This increases the selectivity to the target gases for each sensor in the array. A schematic of the catalytic nanomaterial, embedded into a nanocomposite membrane, interaction with selected gas species is shown in FIG. 2. One of the gas species interact with the catalytic nanofiller and decompose while the other gases permeate through the membrane intact. A schematic nanovoid producing nanomaterial, embedded into a nanocomposite membrane, is shown in FIG. 3. As can be seen, the incorporated nanomaterials change the structural morphology of the nanoscomposite membrane to produce nano-voids that increase the permeativity of the gas species
Electronic circuits 16 consist of a data acquisition system which switches between the sensors, and a coder and modulator that produce the digital data and sends it to the antenna 18 for transmission. Commercial bands (such as 433 MHz) are used for this application as electromagnetic waves in this frequency range can safely penetrate the human tissues. Other commercial bands may be used in various applications. Coding is required to assure that the unique data is sent from each individual capsule. The transmission antenna is a pseudo patch type for transmitting data to the outside of the body data acquisition system. Power source 17 is a battery or super capacitor that can supply the power for the sensors and electronic circuits. A life time of at least 48 hours is required for digestive tract capsules. Generally longer lifetime is needed for other applications.
The dimension of the capsule is preferably less than 1.2 mm in diameter and 3 mm in length, which is swallowable by humans. The body of the capsule is preferably made of indigestible polymer, which is biocompatible. The body is preferably smooth and non-sticky to allow its passage in the shortest possible time and reduces the chances of any capsule retention.

Membrane Preparation

Most preparation methods for nanocomposite membranes primarily involve the mixing of the two major components; the monomer or polymer and the inorganic nano-fillers. Homogenous dispersion of nanofillers within the polymer matrix maximises the benefit those fillers provide to the nanocomposite membranes.

The fabrication methods used are dependent on the form of the organic component (monomer/polymer), and the energy requirements of the mixing and curing processes. It also heavily depends on the type of the inorganic nano-fillers incorporated. In such processes, generally the nano-fillers are made prior to the fabrication of the membranes. Then they are mixed with the monomer or polymer and the membrane is formed through various polymerization and solution evaporation processes. Moreover the membrane framework is another factor that has to be kept in mind when fabricating nano-composite membrane.

If the starter is a monomer then polymerization preferably occurs so that monomer molecules react to form three-dimensional networks of polymer chains around nano-fillers. The chains maybe attached to nano-fillers or make voids around the fillers and depending on the membranes, various pore sizes or nonporous membranes may be obtained. There are many forms of polymerization and different systems exist to categorize them. Polymerization generally takes place via step or chain growth mechanisms. Most of the membrane production mechanisms are based on chain-growth methods. It involves molecules incorporating double or triple carbon-carbon bonds that are linked together in the polymerization process. These monomers have extra internal bonds that can be broken and linked, forming repeating chains. In this case the backbone typically contains carbon atoms. Chain-growth polymerization is involved in the manufacture of polymers such as polyethylene, polypropylene, and polyvinyl chloride (PVC) which are commonly used in the fabrication of gas separation membrane. Similar processes can be adopted using oligomers.

Mixing preparation methods can be divided into the following methods:

Solution blending involves an inorganic solvent that dissolves the polymer and also allows the homogenous dispersion of the nano-fillers. After the dissolution of the polymer component in the solvent, the nano-filler component is added, with thorough, high energy and generally long duration mixing, to allow for uniformity of dispersion. The solutions are then placed into a mold or spread on a surface, and then the solvent is removed, leaving a fully formed nano-composite membrane. Solution blending is one of the simplest methods of nano-composite membrane development. The technique is suitable for a variety of nano-filler types and concentrations as well as polymers. However, the aggregation of nanoparticles within the membranes may be a common issue of this method.

Example: Graphene Nano-Composites

FIG. 4 illustrates the use of graphene nanocomposite membranes. Sensor reading for (a) CH₄ and (b) CO₂ permeation. As can be seen, the pure PDMS response to both 100% CO₂ and CH₄ gases are very long. Graphene nano-composites reduce the response time by producing nano-voids.

The gas permeation mechanisms through these graphene-PDMS nanocomposite membranes differ from other carbon nanomaterial composites. The surface energies of other forms of carbon are very different from those of graphene with no dangling bonds. Carbon fillers, other than graphene, have been used for making permeable composite membranes, generally they have been shown to reduce permeability.

The gas permeation rates of the pristine PDMS and composite graphene-PDMS membranes were investigated under exposure to pure CO₂, N₂, Ar and CH₄ using the constant pressure variable volume (CPVV) experimental setup. As can be seen in FIG. 7, the permeation of all gas species significantly increases with the addition of graphene as a filler to the PDMS matrix.

A maximum permeability for Ar, N₂ and CH₄ was found at 0.25 wt % providing a greatly enhanced flux, over 60% in the case of N₂ for the composite membranes. However, at this condition, there is some minor loss of selectivity, consistent with the Robeson trend of falling selectivity when permeability increases. For CO₂, while the 0.25 wt % graphene-PDMS membrane showed an increase in permeation, it was the 0.5 wt % membrane that provided the greatest flux. Importantly, this increase in permeability is achieved with no loss of CO₂/N₂ selectivity. Indeed, the CO₂/CH₄ selectivity appears to increase slightly.

Gas permeation through a rubbery polymer is dictated by the solution-diffusion mechanism. This mechanism comprises three steps:
(1) adsorption at the upstream boundary,
(2) diffusion through the membrane and
(3) desorption on the downstream boundary.

This difference in the behaviour of CO₂, with greater permeation at 0.5 wt %, whilst the other gas species' maximum permeation occurs at 0.25 wt %, may be ascribed to the high affinity of graphene to CO₂. The increase in permeation for all gases is due to the change in diffusion of the gas molecules through the composite material.

The introduction of graphene into the PDMS matrix increases the amount of free volume within the polymer and thus resulting in an increase in permeation. The presence of graphene in the PDMS matrix has the ability to create permanent voids at these interfaces, where the distance between the oligomers and the graphene flakes is different than the distance between the oligomers themselves under normal crosslinking conditions. The permeation results suggest that there are two separate mechanisms at work altering the gas permeability of the graphene-PDMS membranes. The introduction of extra free volumes through an interfacial void drives an increase in permeability. In contrast, gas transport across the graphene flakes is harder, which naturally decreases the permeability by increasing the diffusion path length for the gas molecules. Therefore considering the two competing effects, the latter may start to dominate at higher wt %. These two effects result in an ‘optimal’ loading concentration.

Example: Antimicrobial Properties of Silver Nano-Composites

While Ag and Ag⁺ ions are useful and effective in bactericidal applications in bulk forms, the unique properties that nanoparticles possess have the potential to enhance any bactericidal effects. Ag nanoparticles display physical properties that are altered from both the ion and the bulk material resulting in an increase in catalytic activity due to an increase in highly reactive facets. If the surface chemistry of Ag nanoparticles is tuned appropriately, they can cause selective toxicity against a wide group of bacteria, while remaining biocompatible for mammalian cells.

Polymers such as, polydimethylsiloxane (PDMS) offer many biomedical and biotechnological applications as well as being utilised in purification technologies. This is due to its many interesting properties: non-toxicity, biocompatibility, optical transparency, durability, flexibility, high permeability to many gas species, hydrophobicity and generally low cost. This makes PDMS a very attractive polymer for being utilized directly or in composite forms. Pure PDMS has been employed for a myriad of applications including implantable devices and biomedical devices as well as being employed extensively throughout many purification processes.

The Ag-PDMS nanocomposite material may show very interesting antibacterial properties with Ag nanoparticle loading within the PDMS matrix, appearing to have significantly reduced the amount of bacteria that adheres to the surface and has decreased the diversity of bacteria growing on the material.

Interestingly, the 0.25 wt % Ag-PDMS nanocomposite showed the least surface coverage or fewest bacterial colonies. This can be ascribed to the maximum concentration of Ag⁺ ions leaching from the nanocomposite which not only affects cells in contact with the surface but those within the surrounding media as well.

Both in vivo and in vitro tests proved that Ag-PDMS nanocomposites, even at relatively low Ag concentrations, show significant antimicrobial properties making it advantageous for biomedical implantable devices.

FIG. 5 illustrates scanning electron microscopy (SEM) images of microbial surface growth from in vivo inside a sheep's rumen investigation on pure PDMS as a reference and Ag PDMS nano-composite of different Ag loading:

(a) pure PDMS at 4 days; (b) 0.25 wt % Ag-PDMS at 4 days; (c) 1 wt % Ag-PDMS at 4 days; (d) pure PDMS at 14 days; (e) 0.25 wt % Ag-PDMS at 14 days; (f) 1 wt % Ag-PDMS at 14 days; (g) pure PDMS at 21 days; (h) 0.25 wt % Ag-PDMS at 21 days and (i) 1 wt % Ag-PDMS at 21 days. As can be seen 0.25 wt % Ag-PDMS nanoscomposite membrane has a remarkable longevity.

Example: Trials were also conducted using membranes with embedded silver in PDMS to measure the reduction of sensor harmful gas species.

Ag-PDMS at 0.25 w/w % Ag reduces the passage of H₂S by 60%

MnO₂-PDMS at 0.5 w/w % MnO₂ reduces the passage of H₂S by 95%

FIG. 6 illustrates a trial of a gas capsule measurement in a pig. This is H₂ profile production on low fibre diet.

Capsules of 1.3 mmx 3.4 mm dimensions were given to pigs. The capsules included a conductometric hydrogen gas sensor. The sensors show large changes after 20 to 30 hours when the capsules transit from the stomach (which is an aerobic environment) to large intestine (which is an anaerobic environment). The also showed signature responses after each feed on low fiber diets. Two peaks were always observed after each feed.

Examples on the performance of nanocomposite membranes at different conditions:

### Example 1

Various nano composite membranes based on rubbery PDMS were trialled with gas sensors as set out in the following table.

The samples are 300 μm thick membranes. All polymers were prepared at the selected conditions to produce the optimum gas permeation.

| | | | Changes in |
|---|---|---|---|
| | | | response with |
| | Base | Nanomaterial used at | reference to |
| Gas species | concentration | 0.25 w/w % | blank PDMS |
| Methane | 1% | MnO₂ (nanomaterials | Completely |
| | | average dimension 100 nm) | blocked for at |
| | | | least 24 hours |
| Methane | 1% | FeOₓ (nanomaterials average | Reduced by |
| | | dimension 85 nm) | ~65% |
| Methane | 1% | CuO nanomaterials average | Almost no |
| | | dimension 110 nm) | change |
| Methane | 1% | MoS₂ (powder-combination | Reduced |
| | | of micro and nano materials) | by 77% |
| Methane | 1% | Graphene | Increased |
| | | | by 60% |
| Hydrogen | 1% | MnO₂ (nanomaterials | Completely |
| | | average dimension 100 nm) | blocked it for at |
| | | | least 24 hours |
| Hydrogen | 1% | FeOₓ (nanomaterials average | Reduced by |
| | | dimension 85 nm) | >95% |
| Hydrogen | 1% | CuO nanomaterials average | Reduced by |
| | | dimension 110 nm) | ~35% |
| Hydrogen | 1% | MoS₂ (powder-combination | No change |
| | | of micro and nano materials) | |
| Hydrogen | 1% | Graphene | Increased by |
| | | | ~83% |
| Carbon | 10% | MnO₂ (nanomaterials | No change |
| dioxiode | | average dimension 100 nm) | |
| Carbon | 10% | FeOₓ (nanomaterials average | No change |
| dioxiode | | dimension 85 nm) | |
| Carbon | 10% | CuO nanomaterials average | No change |
| dioxiode | | dimension 110 nm) | |
| Carbon | 10% | MoS₂ (powder-combination | Increased by |
| dioxiode | | of micro and nano materials) | ~15% |
| Carbon | 10% | Graphene | Increased by |
| dioxiode | | | ~26% |
| Hydrogen | 10 ppm | MnO₂ (nanomaterials | No change |
| disulphide | | average dimension 100 nm) | |
| Hydrogen | 10 ppm | FeOₓ (nanomaterials average | No change |
| disulphide | | dimension 85 nm) | |
| Hydrogen | 10 ppm | CuO nanomaterials average | Completely |
| disulphide | | dimension 110 nm) | blocked |
| Hydrogen | 10 ppm | MoS₂ (powder-combination | Completely |
| disulphide | | of micro and nano materials) | blocked |
| Hydrogen | 10 ppm | Graphene | Nearly the same |
| disulphide | | | |

MnO₂, as a highly active/catalytic nanoparticle, almost fully blocked reactive gas species such as H₂ and CH₄ while had nearly no effect on the permeation of CO₂. It had also no effect on H₂S. FeOₓ was found to be the most effective for blocking H₂. MoS₂ almost had no effect on most of the gas species, while almost completely blocked NO₂. CuO was very effective in blocking H₂S and reducing H₂. While graphene increased the permeation of most of the gas species but had no effect on H₂S.

### Example 2

Nano composite polymer combinations were trialled using noble metals. Although Platinum is not exemplified it is expected that it will perform slightly better than Gold and Silver.

membranes with incorporated Au and Ag nanoparticles with three different model polymers—these are all at 0.25 w/w % of Au and Ag

Polycarbonate was used as a non-rubbery polymer and polyacetylene and polydimethylsiloxane (PDMS) as rubbery polymers

The samples are 300 μm thick membranes. All polymers were prepared at the selected conditions to produce the optimum gas permeation.

| | | | | Effect with |
|---|---|---|---|---|
| | Metal | | | reference to |
| | nanoparticle | | | blank PDMS |
| | and | | Gas | polymer |
| Polymer type | concentration | Gas | concentration | membrane |
| Polycarbonate | Au (80 nm), | CH₄ | 1% | No gas |
| | 0.25 w/w % | | | permeation |
| Polycarbonate | Au (80 nm), | CO₂ | 10% | <0.5% |
| | 0.25 w/w % | | | |
| Polycarbonate | Au (80 nm), | H₂S | 10 ppm | No gas |
| | 0.25 w/w % | | | permeation |
| Polycarbonate | Au (80 nm), | CH₄ | 1% | No gas |
| | 1 w/w % | | | permeation |
| Polycarbonate | Au (80 nm), | CO₂ | 10% | <0.5% |
| | 1 w/w % | | | |
| Polycarbonate | Au (80 nm), | H₂S | 10 ppm | No gas |
| | 1 w/w % | | | permeation |
| Polycarbonate | Ag (80 nm), | CH₄ | 1% | No gas |
| | 0.25 w/w % | | | permeation |
| Polycarbonate | Ag (80 nm), | CO₂ | 10% | <0.1% |
| | 0.25 w/w % | | | |
| Polycarbonate | Ag (80 nm), | H₂S | 10 ppm | No gas |
| | 0.25 w/w % | | | permeation |
| Polycarbonate | Ag (80 nm), | CH₄ | 1% | No gas |
| | 1 w/w % | | | permeation |
| Polycarbonate | Ag (80 nm), | CO₂ | 10% | <0.04% |
| | 1 w/w % | | | |
| Polycarbonate | Ag (80 nm), | H₂S | 10 ppm | No gas |
| | 1 w/w % | | | permeation |
| Polyacetylene | Au (80 nm), | CH₄ | 1% | No gas |
| | 0.25 w/w % | | | permeation |
| Polyacetylene | Au (80 nm), | CO₂ | 10% | ~82% |
| | 0.25 w/w % | | | |
| Polyacetylene | Au (80 nm), | H₂S | 10 ppm | ~36% |
| | 0.25 w/w % | | | |
| Polyacetylene | Au (80 nm), | CH₄ | 1% | No gas |
| | 1 w/w % | | | permeation |
| Polyacetylene | Au (80 nm), | CO₂ | 10% | ~46% |
| | 1 w/w % | | | |
| Polyacetylene | Au (80 nm), | H₂S | 10 ppm | ~28% |
| | 1 w/w % | | | |
| Polyacetylene | Ag (80 nm), | CH₄ | 1% | No gas |
| | 0.25 w/w % | | | permeation |
| Polyacetylene | Ag (80 nm), | CO₂ | 10% | <50% |
| | 0.25 w/w % | | | |
| Polyacetylene | Ag (80 nm), | H₂S | 10 ppm | No gas |
| | 0.25 w/w % | | | permeation |
| Polyacetylene | Ag (80 nm), | CH₄ | 1% | No gas |
| | 1 w/w % | | | permeation |
| Polyacetylene | Ag (80 nm), | CO₂ | 10% | <40% |
| | 1 w/w % | | | |
| Polyacetylene | Ag (80 nm), | H₂S | 10 ppm | No gas |
| | 1 w/w % | | | permeation |
| PDMS | Au (80 nm), | CH₄ | 1% | No change |
| | 0.25 w/w % | | | |
| PDMS | Au (80 nm), | CO₂ | 10% | No change |
| | 0.25 w/w % | | | |
| PDMS | Au (80 nm), | H₂S | 10 ppm | ~30% |
| | 0.25 w/w % | | | decrease |
| PDMS | Au (80 nm), | CH₄ | 1% | ~5% change |
| | 1 w/w % | | | |
| PDMS | Au (80 nm), | CO₂ | 10% | ~30% |
| | 1 w/w % | | | decrease |
| PDMS | Au (80 nm), | H₂S | 10 ppm | ~20% |
| | 1 w/w % | | | decrease |
| PDMS | Ag (80 nm), | CH₄ | 1% | No change |
| | 0.25 w/w % | | | |
| PDMS | Ag (80 nm), | CO₂ | 10% | ~14% |
| | 0.25 w/w % | | | decrease |
| PDMS | Ag (80 nm), | H₂S | 10 ppm | ~76% |
| | 0.25 w/w % | | | decrease |
| PDMS | Ag (80 nm), | CH₄ | 1% | No change |
| | 1 w/w % | | | |
| PDMS | Ag (80 nm), | CO₂ | 10% | ~40% |
| | 1 w/w % | | | decrease |
| PDMS | Ag (80 nm), | H₂S | 10 ppm | ~60% |
| | 1 w/w % | | | decrease |

Polycarbonate was almost non-permeative to most of the gas species, while both rubbery polyacetylene and PDMS show high degrees of permeation. PDMS was certainly a better gas permeative material for all gas species tested.

### Example 3

nano-composite polymer combinations for nano particles MnO₂, FeOₓ, CuO, WS₂, and MoS₂ were trialled using a model binary compound of polyacetylene and PDMS at 50 w/w % each.

The samples are 300 μm thick membranes. All polymers were prepared at the selected conditions to produce the optimum gas permeation.

| | | | Changes in |
|---|---|---|---|
| | Base | | response with |
| | con- | Nanomaterial used | reference to blank |
| Gas species | centration | at 0.25 w/w % | PDMS |
| Methane | 1% | MnO₂ (nanomaterials | Completely blocked |
| | | average dimension | for at least 24 hours |
| | | 100 nm) | |
| Methane | 1% | FeOₓ (nanomaterials | Reduced by ~79% |
| | | average dimension | |
| | | 85 nm) | |
| Methane | 1% | CuO nanomaterials | Reduced by ~87% |
| | | average dimension | |
| | | 110 nm) | |
| Methane | 1% | MoS₂ (powder- | Reduced by ~54% |
| | | combination of micro | |
| | | and nano materials) | |
| Methane | 1% | Graphene | Increased by ~5% |
| Hydrogen | 1% | MnO₂ (nanomaterials | Completely blocked |
| | | average dimension | it for at least 24 |
| | | 100 nm) | hours |
| Hydrogen | 1% | FeOₓ (nanomaterials | Completely blocked |
| | | average dimension | |
| | | 85 nm) | |
| Hydrogen | 1% | CuO nanomaterials | Completely blocked |
| | | average dimension | |
| | | 110 nm) | |
| Hydrogen | 1% | MoS₂ (powder- | Reduced by ~33% |
| | | combination of micro | |
| | | and nano materials) | |
| Hydrogen | 1% | Graphene | Increased by ~10% |
| Carbon dioxiode | 10% | MnO₂ (nanomaterials | No change |
| | | average dimension | |
| | | 100 nm) | |
| Carbon dioxiode | 10% | FeOₓ (nanomaterials | No change |
| | | average dimension | |
| | | 85 nm) | |
| Carbon dioxiode | 10% | CuO nanomaterials | No change |
| | | average dimension | |
| | | 110 nm) | |
| Carbon dioxiode | 10% | MoS₂ (powder- | No change |
| | | combination of micro | |
| | | and nano materials) | |
| Carbon dioxiode | 10% | Graphene | Increased by ~12% |
| Hydrogen | 10 ppm | MnO₂ (nanomaterials | Completely |
| disulphide | | average dimension | blocked |
| | | 100 nm) | |
| Hydrogen | 10 ppm | FeOₓ (nanomaterials | Completely |
| disulphide | | average dimension | blocked |
| | | 85 nm) | |
| Hydrogen | 10 ppm | CuO nanomaterials | Reduced by ~28% |
| disulphide | | average dimension | |
| | | 110 nm) | |
| Hydrogen | 10 ppm | MoS₂ (powder- | Completely |
| disulphide | | combination of micro | blocked |
| | | and nano materials) | |
| Hydrogen | 10 ppm | Graphene | Increased by ~73% |
| disulphide | | | |

It seems that introducing binary compounds tend to have no effect or reduce the overall permeation for most of the gas species in comparison to pure PDMS except for H₂S gas molecules. Interestingly the permeation rate of H₂S, which is a relatively larger gas molecule in comparison to H₂, CH₄ and CO₂ increased. It seems that making the binary compound favours the permeation of larger molecules by producing relatively larger pores between the polymer chains.

The digestive system gas capsules with nanocomposite membranes can be potentially modified to be used for other applications. This includes those for some areas of mining sectors and farming as well as environmental pollution that especially concern water contamination. A large number of these capsules can be distributed across fields to collect the information about the gas constituents in air or water. Capsules with the array of sensors can send the gas data, depending on the transmission range of the system. The nanocomposite membranes will help in the accuracy of the measurements by making the system more selective, increasing the longevity of the system by blocking harmless gases (or possible colonization of bacterial components in the environment) and reducing the response time (using nanovoid membranes) to obtain correct gas measurements at the smallest buttery power consumption. In such cases the capsule systems should transmit coded data to allow the unique data transfer from each sensor.

Those skilled in the art will realise that this invention provides a valuable contribution to diagnosis of disorders in the mammalian digestive system. It also generates information about the health status of mammalians and gas production in their digestive system. Those skilled in the art will also realise that this invention may be implemented in embodiments other than those described without departing from the core teachings of this invention.

1. (canceled)

2. The capsule of claim 9, wherein the polymer film is formed from a polymer selected from the group consisting of polycarbonate, polydimethylsiloxane and polyacetylene.

3. The capsule of claim 9, wherein the gas permeable, liquid impermeable membrane has reduced permeability to hydrogen and methane in comparison with a membrane that is the same but without the nanoparticles.

4. The capsule of claim 3, wherein the one or more kinds of nanoparticles include MnO2 nanoparticles.

5. The capsule of claim 9, wherein the gas permeable, liquid impermeable membrane has reduced permeability to hydrogen and hydrogen disulfide in comparison with a membrane that is the same but without the nanoparticles.

6. The capsule of claim 5, wherein the one or more kinds of nanoparticles include FeOx and/or CuO nanoparticles.

7. The capsule of claim 9, wherein the one or more kinds of nanoparticles include graphene nanoparticles.

8. The capsule of claim 9, wherein the one or more kinds of nanoparticles include silver, gold or platinum nano-particles.

9. A capsule adapted to be introduced into the digestive system and gastrointestinal (GI) tract of a mammal which consists of capsule shaped comprising:
a container including a wall comprising an indigestible, biocompatible polymer and being adapted to house at least one electronic or sensor device contained within the capsule;
the wall including at least one gas permeable liquid membrane;
wherein the membrane comprises a polymer film incorporating one or more kinds of nanoparticles, the nanoparticles producing interfacial voids in the polymer film, wherein one or more of the following properties of the membrane are improved in comparison with a membrane that is the same but without the nanoparticles:
inhibition of microbial growth on the membrane;
gas permeability; and
gas selectivity;
wherein said inhibition of microbial growth on the membrane is improved due to the presence of the nanoparticles; and
either or both of gas permeability and gas selectivity are improved due to the production of interfacial voids in the polymer film.
